(19) United States
(12) Patent Application Publication
(10) Pub. No.: US 2016/0291005 A1
(43) Pub. Date: Oct. 6, 2016

(54) SENSORS HAVING INTERNAL CALIBRATION OR POSITIVE CONTROLS

(71) Applicant: RAPID DIAGNOSTEK, INC., Plymouth, MN (US)

(72) Inventors: Michael Salvati, Minnetrista, MN (US); Ian Robert Harmon, Minneapolis, MN (US)

(21) Appl. No.: 15/035,552

(22) PCT Filed: Nov. 12, 2014
(86) PCT No.: PCT/US14/65151

§ 371 Date: May 10, 2016

Related U.S. Application Data
(60) Provisional application No. 61/903,326, filed on Nov. 12, 2013.
(60) Provisional application No. 61/906,127, filed on Nov. 19, 2013.
(60) Provisional application No. 61/906,030, filed on Nov. 19, 2013.

(51) Int. Cl. G01N 33/543

(52) U.S. Cl. CPC G01N 33/54373; G01N 2333/4712

(57) ABSTRACT

Analyte-detecting sensor devices include a test sensor, a reference sensor and one or more positive control or calibration sensors. The sensors are in fluid communication with each other. For example, the sensors may be positioned in and in fluid communication with a flow path of the sensor device.

### RELATED APPLICATIONS

This application claims the benefit of U.S. Provisional Patent Application No. 61/903,326, filed on Nov. 12, 2013, U.S. Provisional Patent Application No. 61/906,030, filed on Nov. 19, 2013, and U.S. Provisional Patent Application No. 61/906,127, filed on Nov. 19, 2013, each of which provisional patent applications are hereby incorporated herein by reference in their respective entireties to the extent that they do not conflict with the disclosure presented herein.

### FIELD

This disclosure relates to, among other things, analyte-detecting sensor devices, such as immunosensor devices, having positive controls or internal calibration capabilities.

### SUMMARY

This disclosure relates to, among other things, analyte-detecting sensor devices having a test sensor and one or more positive control or calibration sensors. In some embodiments, the sensors are in fluid communication with each other. For example, the sensors may be positioned in a sample flow path. Placement of positive control or calibration sensors in fluid communication with the test sensors can provide for increased assay efficiency. However, the use of one flow path across all sensors may present concerns with highly sensitive sensors or assays, because the risk of indiscriminant or unintended effect at one or more sensors can be large. In some embodiments, the sensor devices employ thin film bulk acoustic resonators (TFBARs) operating at high frequencies with signal amplification. Even with such systems, efficient and accurate analyte detection can occur when the test sensor and the one or more positive control or calibration sensors are in fluid communication.

In some embodiments, a device for detecting an analyte in a sample includes (i) one or more test sensors configured to bind the analyte; (ii) optionally one or more reference sensors configured to not bind the analyte; (iii) one or more control sensor comprising a known amount of bound analyte or analyte analog, which can be different for each control sensor; and (iv) a fluid flow path for carrying the sample. The one or more test sensors, the one or more reference sensors, if present, and the one or more control sensors are positioned along and in communication with the fluid flow path. The device further includes control electronics operably coupled to the one or more test sensors, the one or more reference sensors, if present, and the one or more control sensors. The control electronics are configured to compare signals generated from the one or more test sensors, the one or more reference sensors, if present, and the one or more control sensors to determine an amount of analyte present in the sample. In some embodiments, the sensors comprise thin film bulk acoustic resonators.

In some embodiments, a sensor assembly for use with a device for detecting an analyte in a sample is described herein. The device has a flow path through which the sample is configured to flow. The sensor assembly includes (i) one or more test sensors each comprising a thin film bulk acoustic resonator (TFBAR) having a surface to which an analyte-binding partner is immobilized; (ii) optionally one or more reference sensors each comprising a TFBAR having a surface to which an analyte-nonbinding partner is immobilized; and (iii) one or more control sensors each comprising a TFBAR having a surface to which the analyte or the analyte analog is immobilized. The sensor assembly is configured to be operably coupled with the device for detecting the analyte in the sample. The sensor assembly is configured to be at least partially inserted into the device such that the one or more test sensors, the one or more reference sensors, if present, and the one or more control sensors are positioned in and in communication with the flow path of the device.

In some embodiments, a method for determining an amount of an analyte in a sample includes (A) introducing (i) the sample or (ii) the sample and a tag-linked analyte molecule to a fluid flow path disposed across at least a portion of one or more test sensor, one or more optional reference sensors, if present, and one or more control sensors, (B) measuring an amount, or indicator thereof, of the analyte, the tag-linked analyte, or the tag bound to the one or more test sensors, the one or more reference sensors, if present, and the one or more control sensors; and (C) comparing the amount of analyte, tag-linked analyte, tag or indicator thereof bound to the one or more test sensors, the one or more reference sensors, if present, and the one or more control sensors to determine the amount of analyte in the sample. The one or more test sensors, in the flow path, each comprise a surface to which an analyte-binding partner is immobilized. If present, the one or more reference sensors, in the flow path, each comprise a surface to which an analyte-nonbinding partner is immobilized. The one or more control sensors, in the flow path, each comprise a surface to which the analyte, the tag-linked analyte molecule, an analyte analog, a tag-linked analyte analog, or a tag is immobilized.

In various embodiments of the methods, devices, systems, etc. described herein, the analyte is troponin I (TnI).

One or more embodiments of the sensors, devices, systems or methods described herein provide one or more advantages over prior sensors, devices, systems or methods for detecting small quantities of an analyte. Such advantages will be readily understood by those of skill in the art from the following detailed description.

### BRIEF DESCRIPTION OF THE DRAWINGS

FIG. 1 is a schematic drawing of an embodiment of a sensor system.

FIGS. 2A-C are schematic drawings illustrating principles, circuitry and components of embodiments of thin film bulk acoustic resonator sensors.

FIGS. 3A-B are schematic drawings of an embodiment of a sensor cartridge configured to be received by an embodiment of a sensor device.

FIG. 4 is a schematic drawing of an embodiment of a sensor assembly including a test sensor, a reference sensor and two control sensors.

FIG. 5 is a schematic drawing of an embodiment of an assay scheme, or portion thereof, for detecting an analyte employing an embodiment of a sensor assembly.

FIG. 6A-B are schematic drawings of an embodiment of a test sensor illustrating selective binding of an analyte.

FIGS. 7A-B are schematic drawings of an embodiment of a reference sensor illustrating that the sensor is configured to not bind analyte.

FIGS. 8A-B are schematic drawings illustrating signal generating or enhancing-linked conjugate analyte binding partner is configured to bind either or both of analyte or an analyte analog or derivative.

FIG. 9 is a schematic drawing of an embodiment of an assay scheme, or portion thereof, for detecting an analyte employing an embodiment of a sensor assembly.

FIG. 10 is a schematic drawing illustrating an embodiment of an enzyme-mediated mass loading on a test sensor.

FIG. 11 is a schematic drawing of an embodiment of an assay scheme, or portion thereof, for detecting an analyte employing an embodiment of a sensor assembly.

FIG. 12 is a schematic drawing of an embodiment of an assay scheme, or portion thereof, for detecting an analyte employing an embodiment of a sensor assembly.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description several specific embodiments of devices, systems, kits and methods are disclosed, It is to be understood that other embodiments are contemplated and may be made without departing from the scope or spirit of the present disclosure. The following detailed description, therefore, is not to be taken in a limiting sense.

This disclosure generally relates to, among other things, methods, devices, sensors and systems for detecting an analyte. The sensor systems and devices described herein include one or more test sensors and one or more positive control or calibration sensors. In some embodiments, the sensor systems and devices described herein can include, but need not include, one or more reference sensors that are configured to not bind an analyte, analyte analog, etc. In some embodiments, the sensors are in fluid communication with each other.

Any suitable sensor device can be modified to include one or more test sensors, one or more optional reference sensors, and one or more control sensors in a fluid flow path. In some embodiments, the sensor device is a device as described in PCT Patent Application PCT/US2014/039400, entitled TWO PART ASSEMBLY, filed on May 23, 2014, naming Rapid Diagnostek as applicant, or a modified version of such a device that includes one or more test sensors, optionally one or more reference sensors, and one or more control sensors in a fluid flow path. PCT Patent Application PCT/US2014/039400 is hereby incorporated herein by reference to the extent that it does not conflict with the disclosure presented herein. In some embodiments, the sensor device is a colorimetric, fluorescence-detecting, isotope-detecting, light-detecting, or other suitable sensor device.

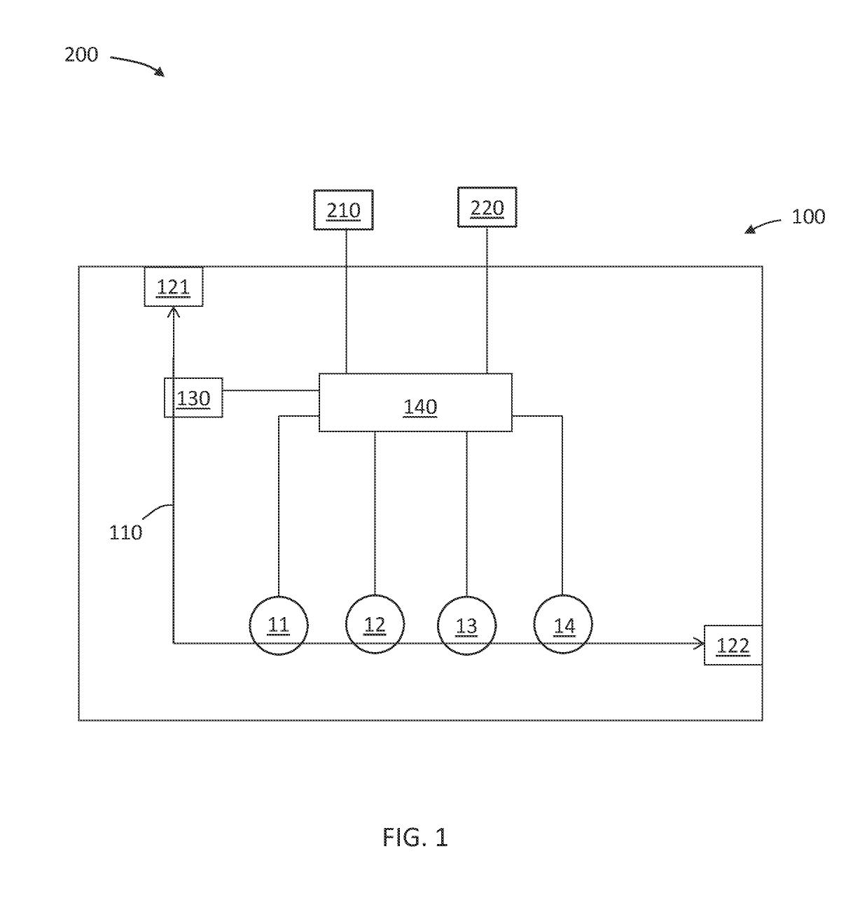

Regardless of the type of sensor or sensor device, each of the sensors can be disposed in the path of a microfluidic channel configured to carry a test sample or reagents, which are preferably in liquid form for flow through the channel. For example and with reference to FIG. 1, a schematic drawing of an embodiment of sensor system 200 is shown. In the embodiment depicted in FIG. 1, the system 200 includes a sensor device 100 that has a test sensor 11, a reference sensor 12 and two control or calibration (which will be referred to hereinafter as a “control”) sensors 13, 14. At least a portion of each of the sensors 11, 12, 13, 14 is in a flow path 110 configured to carry a test sample and optionally one or more assay reagents from an input port 122 to an exit port 121. In some embodiments, the flow path 110, or at least a portion in which the sensors 11, 12, 13, 14 are disposed, can be a microfluidic path. One or more optional pumps 130 or other flow control device can be placed in flow path 110 to control movement of fluid through the device 100. The flow of sample may be unidirectional (input to output) or multi-directional (e.g., back and forth across sensors).

In some embodiments (not shown), the input port 122 and the output port 121 are the same port. In some embodiments (not shown), multiple input ports can be in communication with and feed into flow path 110. For example, one input port may be used to introduce a sample and another may be used to introduce one or more assay reagent.

Still referring to FIG. 1, the device 100 includes control electronics 140 operably coupled to the one or more optional pumps 130 and to each sensor 11, 12, 13, 14. Control electronics 140 are depicted as being discretely bound to sensors 11, 12, 13, 14, but may be multiplexed in some embodiments. Regardless of details of operable connection, control electronics 140 are configured to compare test sensor 11 signals to optional reference sensor 12 signals and control sensors 13, 14 signals and to determine an amount of analyte present in a sample.

The test sensor 12 is configured to bind the analyte; the optional reference sensor is configured not to bind the analyte; and the control sensors 13, 14 include a known amount of bound analyte, analyte analog, or the like (which may be present in a different amount for each control sensor). If more than one control sensors, each with different amounts of analyte, analyte analog, etc. bound, are employed, control electronics can develop a calibration curve based on signal from the control sensors to determine the amount of analyte in the sample. By way of example, control electronics 140 can be configured to subtract a signal obtained from reference sensor 12 from a signal obtained from test sensor 11 and to compare the subtracted signal to signals obtained from control sensors 13, 14 to determine the amount of analyte in the sample. Alternatively or in addition, the signal from the reference sensor and the control sensors may be used to develop a calibration curve (e.g., zero, low and high) against which signal from the test sensor may be compared to determine the amount of analyte in the sample. In some embodiments, a reference sensor is not employed to determine the amount of analyte in the sample.

Signal obtained from the one or more control sensors 13, 14, as well as the optional reference sensor 12, can be used as internal quality control for each assay as the signal generated on each should fall with defined limits (e.g., predetermined signal magnitude based on amounts of bound analyte that can be stored in memory of control electronics). Failure of control sensors responses to fall within predetermined limits could be used to warn of potentially erroneous results or even prevent a result from being reported.

In some embodiments, the capacity of the test sensor 11 to bind the analyte is in excess of the amount of analyte that can bind the test sensor during a portion of an assay in which data is collected. Typically, data is collected during an initial period of binding (soon after the sample is introduced to the sensor). During the initial binding period binding kinetics can more readily be determined, as opposed to, for example, at equilibrium. The sample may be diluted, as needed or desired, to achieve an amount of analyte present within a range to allow meaningful comparison of a test sensor signal with signals from the one or more control sensors.

Control electronics 140 can include any suitable components for operation of sensor device 100 or system 200. Control electronics can include a processor, memory, sensor actuation circuitry, measurement circuitry, and the like. The memory can contain computer-readable instructions that, when executed by the processor, cause the device 100 and processor to perform various functions attributed to the device and processor herein. Memory can include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

A processor may include any one or more of a microprocessor, a controller, a digital signal processor (DSP), an application specific integrated circuit (ASIC), a field-programmable gate array (FPGA), or equivalent discrete or integrated logic circuitry. In some examples, a processor can include multiple components, such as any combination of one or more microprocessors, one or more controllers, one or more DSPs, one or more ASICs, or one or more FPGAs, as well as other discrete or integrated logic circuitry. The functions attributed to a processor herein may be embodied as software, firmware, hardware or any combination thereof. A processor can control sensor actuation circuitry, if included, to actuate or activate sensors 11, 12, 13, 14, measure signals from the sensors and compare the signals to determine an amount of analyte present in a sample. The actuation and measurement circuitry (and associated components) can depend on the type of sensor employed. For example, if the sensor is a fluorescence sensor, the activation circuitry may include circuitry (and other suitable components) configured to emit energy at a wavelength suitable to elicit fluorescence (excitation energy) and measurement circuitry can include a detector configured to detect energy at a wavelength emitted from excited fluorescent molecules. Other actuation and measurement circuitry (and related components) for other sensor schemes will be readily understood to those of skill in the art. For purposes of example, some embodiments of actuation and sensing schemes and circuitry for use with thin-film bulk acoustic resonators (TFBARs) will be discussed below in more detail.

Device 100 can include a power source (not shown), which can be, for example, an AC or DC power source that is operably coupled to control electronics 140, pump 130 and other components of device.

System 200 may include input apparatus 210 and display apparatus 220 operably coupled to control electronics 140. Any suitable input apparatus 210, such as a touch screen, keyboard, etc., may be employed. Any suitable display apparatus 220, such as a monitor, touchscreen, or the like may be employed. In some embodiments, input apparatus 210 and display apparatus 220 are the same or share one or more common components; e.g., where input apparatus and display apparatus are a touchscreen. Control electronics 140 may be configured to receive input from input apparatus 220 and transmit output to display apparatus 222. For example, output regarding the amount of analyte determined to be present in a sample may be displayed on output apparatus 220

In view of the above, it will be readily apparent that the functionality as described in one or more embodiments according to the present disclosure may be implemented in any manner as would be known to one skilled in the art. As such, the computer language, the computer system, or any other software/hardware which is to be used to implement the processes described herein shall not be limiting on the scope of the systems, processes or programs (e.g., the functionality provided by such systems, processes or programs) described herein.

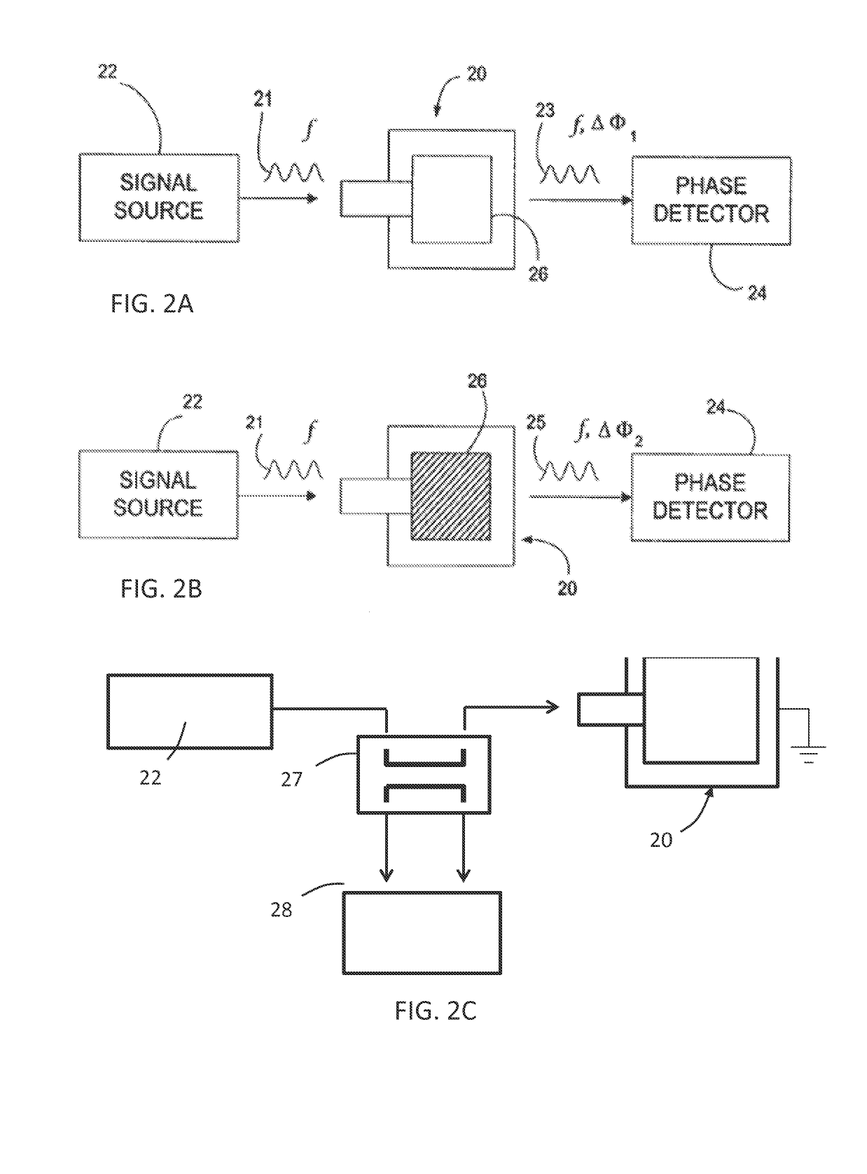

Referring now to FIGS. 2A-B, general operating principles of an embodiment of a bulk-acoustic wave piezoelectric resonator 20 used as a sensor to detect an analyte are shown. The resonator 20 typically includes a planar layer of piezoelectric material bounded on opposite sides by two respective metal layers which form the electrodes of the resonator. The two surfaces of the resonator are free to undergo vibrational movement when the resonator is driven by a signal within the resonance band of the resonator. When the resonator is used as a sensor or incorporated into a sensor, at least one of its surfaces is adapted to provide binding sites for the material being detected. The binding of the material on the surface of the resonator alters the resonant characteristics of the resonator, and the changes in the resonant characteristics are detected and interpreted to provide quantitative or semi-quantitative information regarding the material being detected.

By way of example, such quantitative information may be obtained by detecting a change in the insertion or reflection coefficient phase shift of the resonator caused by the binding of the material being detected on the surface of the resonator. Such sensors differ from those that operate the resonator as an oscillator and monitor changes in the oscillation frequency. Rather such sensors insert the resonator in the path of a signal of a pre-selected frequency and monitor the variation of the insertion or reflection coefficient phase shift caused by the binding of the material being detected on the resonator surface. Of course, sensors that monitor changes in oscillation frequency may also be employed in accordance with signal amplification described herein.

In more detail, FIG. 2A shows the resonator 20 before the material being detected is bound to its surface 26. The depicted resonator 20 is electrically coupled to a signal source 22, which provides an input electrical signal 21 having a frequency f within the resonance band of the resonator. The input electrical signal is coupled to the resonator 20 and transmitted through the resonator to provide an output electrical signal 23. In the depicted embodiment, the output electrical signal 23 is at the same frequency as the input signal 21, but differs in phase from the input signal by a phase shift ΔΦ₁, which depends on the piezoelectric properties and physical dimensions of the resonator. The output signal 23 is coupled to a phase detector 24 which provides a phase signal related to the insertion phase shift.

FIG. 28 shows the sensing resonator 20 with the material being detected bound on its surface 26. The same input signal is coupled to the resonator 20. Because the resonant characteristics of the resonator are altered by the binding of the material as a perturbation, the insertion phase shift of the output signal 25 is changed to ΔΦ₂. The change in insertion phase shift caused by the binding of the material is detected by the phase detector 24. The measured phase shift change is related to the amount of the material bound on the surface of the resonator.

FIG. 2C shows an alternative to measuring the insertion phase of the resonator. A directional coupler 27 is added between the signal source 22 and the resonator 20 with the opposite electrode grounded. A phase detector 28 is configured to measure the phase shift of the reflection coefficient as a result of material binding to the resonator surface.

Other TFBAR phase-shift sensors that may be employed with the signal amplification aspects described herein include those described in, for example, U.S. Pat. No. 8,409,875 entitled “RESONATOR OPERATING FREQUENCY OPTIMIZATION FOR PHASE-SHIFT DETECTION SENSORS,” which patent is hereby incorporated herein by reference in its entirety to the extent that it does not conflict with the disclosure presented herein. For example, sensor apparatuses may include (i) a sensing resonator comprising binding sites for an analyte, a reference resonator that does not contain binding sites for the analyte, and one or more control sensor containing bound analyte, analyte analog, etc.; (ii) actuation circuitry configured to drive the resonators in an oscillating motion; (iii) measurement circuitry arranged to be coupled to the resonators and configured to measure one or more resonator output signals representing resonance characteristics of the oscillating motion of the resonators; and (iv) a controller operatively coupled with the actuation and measurement circuitry. The controller can be interfaced with data storage containing instructions that, when executed, cause the controller to adjust the frequency at which the actuation circuitry drives the resonators to maintain a resonance point of the resonators. Accordingly, sensing may be accomplished by actuating the TFBARs into an oscillating motion; measuring one or more resonator output signals representing resonance characteristics of the oscillating motion of the TFBARs; and adjusting the actuation frequency of the resonators to maintain a resonance point of the TFBARs. In some embodiments, the frequency at which the actuation circuitry drives the resonators is a frequency of maximum group delay.

Such phase detection approaches can be advantageously used with piezoelectric resonators of different resonant frequencies.

In various embodiments, TFBARs for use with the methods, devices, and system described herein have resonance frequencies of about 500 MHz or greater, such as about 700 MHz or greater, about 900 MHz or greater, about 1 MHz or greater, 1.5 GHz or greater, about 1.8 GH or greater, about 2 GHz or greater, 2.2 GHz or greater, 2.5 GHz or greater, about 3 GHZ or greater, or about 5 GHZ or greater can provide enhanced sensitivity when used with amplification element mediated mass loaded, which is described in more detail below. In some embodiments, the TFBARs have resonance frequencies of from about 500 MHz to about 5 GHz, such as from about 900 MHz to about 3 GHz, or from about 1.5 GHz to about 2.5 GHz. Some of such frequencies are substantially higher than frequencies of previously described piezoelectric resonators.

Some embodiments of the resonators described herein are thin-film resonators. Thin film resonators comprise a thin layer of piezoelectric material deposited on a substrate, rather than using, for example, AT-cut quartz. The piezoelectric films typically have a thickness of less than about 5 micrometers, such as less than about 2 micrometers, and may have thicknesses of less than about 100 nanometers. Thin-film resonators are generally preferred because of their high resonance frequencies and the theoretically higher sensitivities. Depending on the applications, a thin-film resonator used as the sensing element may be formed to support either longitudinal or shear bulk-acoustic wave resonant modes. Preferably, the sensing element is formed to support shear bulk-acoustic wave resonant modes, as they are more suitable for use in a liquid sample.

Additional details regarding sensor devices and systems that may employ TFRs are described in, for example, U.S. Pat. No. 5,932,953 issued Aug. 3, 1999 to Drees et al., which patent is hereby incorporated herein by reference in its entirety to the extent that it does not conflict with the disclosure presented herein.

TFR sensors may be made in any suitable manner and of any suitable material. By way of example, a resonator may include a substrate such as a silicon wafer or sapphire, a Bragg mirror layer or other suitable acoustic isolation means, a bottom electrode, a piezoelectric material, and a top electrode.

Any suitable piezoelectric material may be used in a TFR. Examples of suitable piezoelectric substrates include lithium tantalate (LiTaO₃), lithium niobate (LiNbO₃), Zinc Oxide (ZnO), aluminum nitride (AlN) plumbum zirconate titanate (PZT) and the like.

Electrodes may be formed of any suitable material, such as aluminum, tungsten, gold, titanium, molybdenum, or the like. Electrodes may be deposited by vapor deposition or may be formed by any other suitable process.

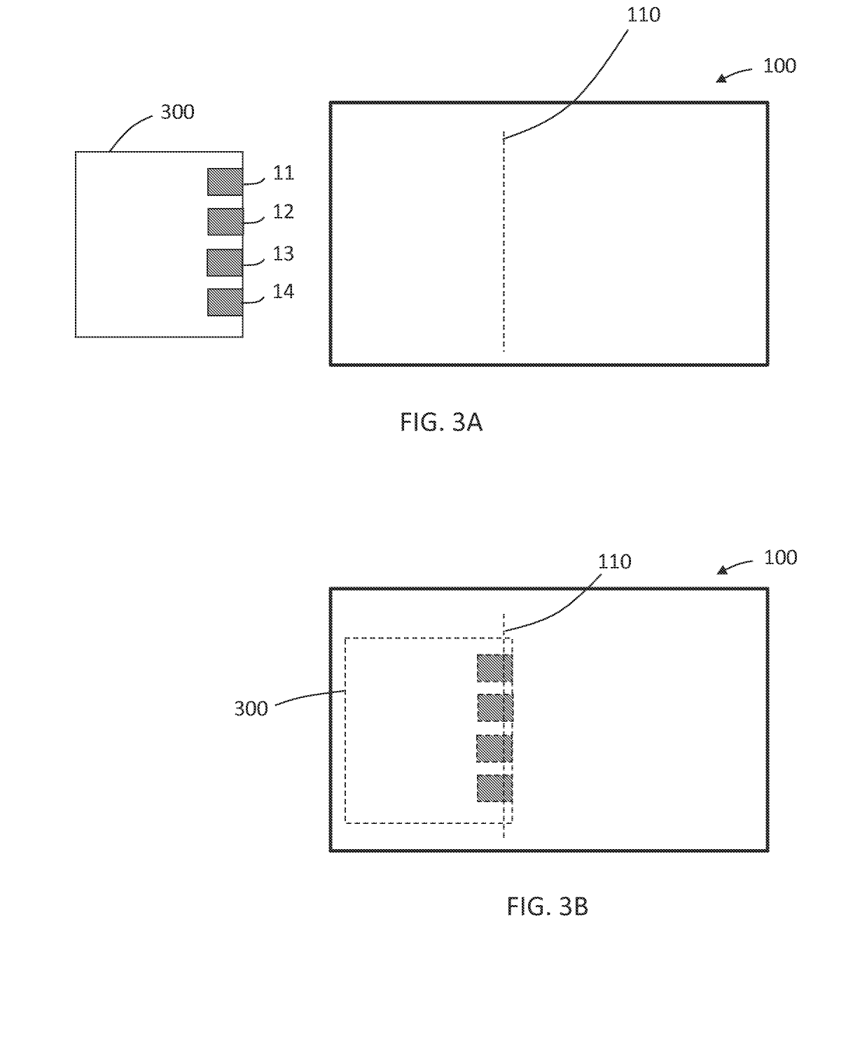

Regardless of the type of sensor employed, the sensors may be included in a cartridge tier insertion into a sensor device. For example and with reference to FIGS. 3A-B, a cartridge 300 including one or more test sensors 11 (one depicted), one or more reference sensor 12 (one depicted) and one or more control sensors 13, 14 (two depicted) is shown. The cartridge 300 is configured to be received by a sensor device 100 such that at least a portion of the sensors 11, 12, 13, 14 are positioned in flow path 110 of device 100. The cartridge 300 may include suitable components for operably coupling the sensors 11, 12, 13, 14 to control electronics of device 100. In some embodiments the cartridge 300 includes a printed circuit board with which sensors 11, 12, 13, 14 are operably coupled. The printed circuit board may include contacts for operably coupling sensors 11, 12, 13, 14 to control electronics of device 100. In some embodiments, cartridge 100 is a cartridge as described in PCT Patent Application PCT/US2014/039294, entitled interconnect Device and Module Using Same, filed on May 23, 2014, and naming Rapid Diagnostek as applicant, which PCT application is hereby incorporated herein by reference in its entirety to the extent that it does not conflict with the disclosure presented herein.

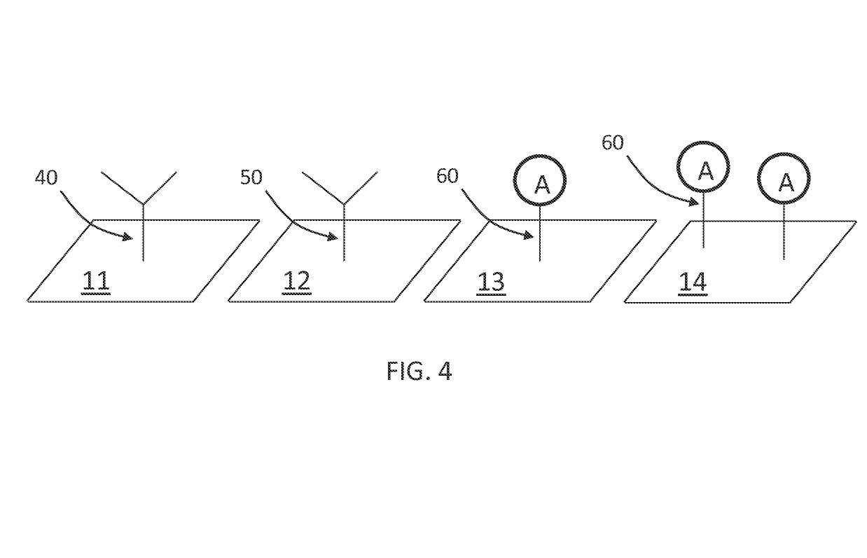

Referring now to FIG. 4, a schematic drawing of an embodiment of a test sensor 11, a reference sensor 12, a first control sensor 13 and a second control sensor 14 that may be employed in combination is depicted. As discussed above, any number of test sensors, optional reference sensors, and control sensors can be employed (which is also true for other embodiments discussed above or below). Each sensor 11, 12, 13, 14 has a surface. In the depicted embodiment, the test sensor 11 has a capture analyte-binding partner 40, such as an antibody directed to an analyte, bound (covalently or non-covalently) thereto. The reference sensor 12 has a molecule 50 that is similar (preferably as similar as possible) to the capture analyte-binding partner 40, but which does not selectively bind the analyte, bound thereto. The control sensors 13, 14 (in this case there are two control sensors, but there may be any suitable number of one or more control sensors) having a surface to which an analyte A is bound. The analyte A may be bound to the surface in any suitable manner, either directly to the sensor surface or via a linker molecule 60, such as bovine serum albumin, an irrelevant antibody (e.g., an irrelevant IgG antibody), or the like. In the depicted embodiment, control sensor 13 has a different (lower or higher) concentration of analyte A bound to its surface than control sensor 14.

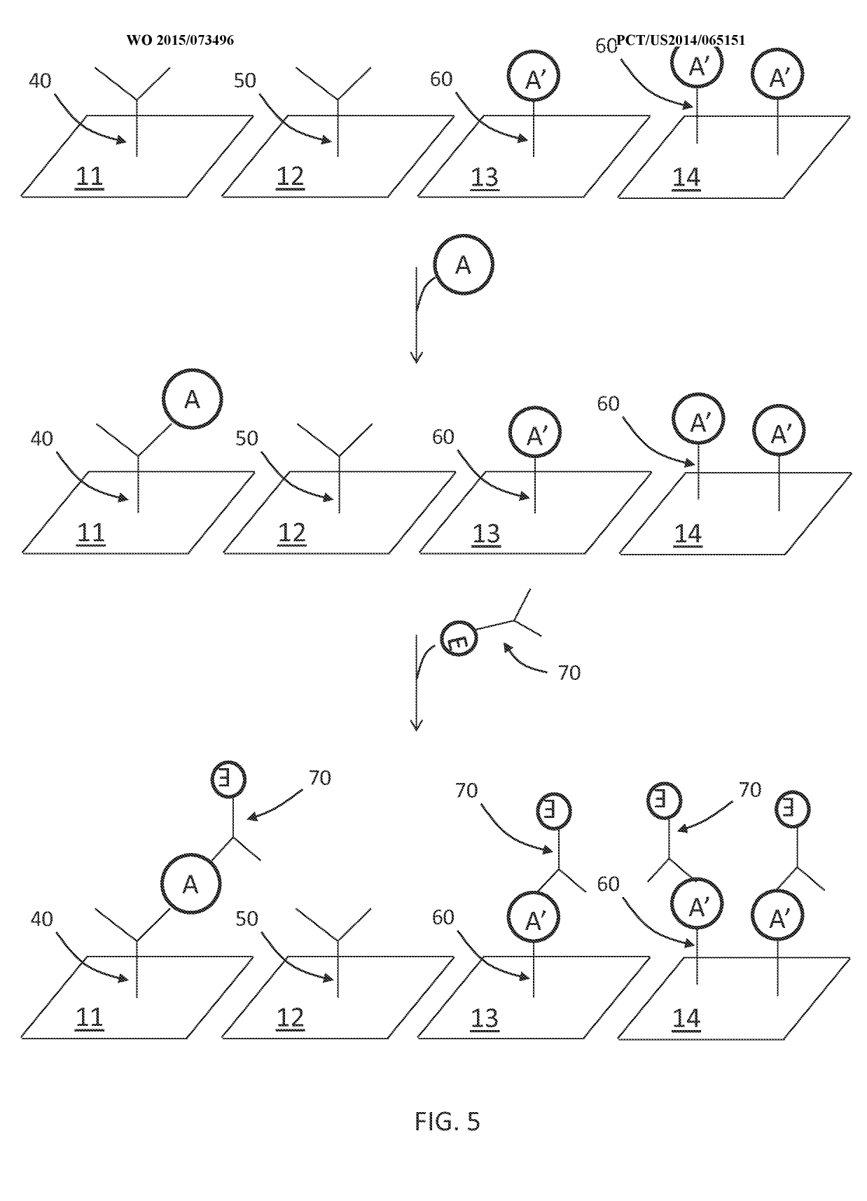

FIG. 5 is a schematic drawing of an embodiment of an assay employing an embodiment of a test sensor 11, reference sensor 12, first control sensor 13 and second control sensor 14. As illustrated in the top panel of FIG. 5, the control sensors 13, 14 may contain an analyte analog (molecule A′), which is different from analyte A, rather than analyte A. Molecule A′ does not selectively bind to capture analyte-binding partner 40, but does selectively bind to conjugate analyte-binding partner (as will be discussed below). Molecule A′ may be, for example, a variant of analyte A, such as a specific or altered epitope of analyte A (e.g., analyte A but having an altered epitope).

Still with reference to FIG. 5, sensors 11-14 may be exposed to a test sample that may contain analyte A (e.g., test sample may be introduced through microfluidic channel 80 in a device as depicted in FIG. 1). As shown in the middle panel of FIG. 5, analyte A may bind selectively with capture analyte-binding partner 40 on test sensor 11, but not analyte non-binding partner 50 that is similar to the capture analyte-binding partner 40 on reference sensor 12. Analyte A (preferably has no selective interaction with linker molecule 60. The sensor surfaces may be washed and conjugate analyte-binding partner 70, such as an antibody conjugated to an enzyme or other signaling moiety or signal facilitating moiety (moiety E, also refined to herein as “signal enhancement element”). Of course, the analyte A and conjugate analyte-binding partner 70 may be added at the same time.

As shown in the bottom panel of FIG. 5, conjugate analyte-binding partner 70 selectively binds to both analyte A and molecule A′. The conjugate analyte-binding partner 70 provides a signal or aids in providing a quantifiable signal that correlates to amount of analyte A (or molecule A′) bound to test sensor 11 (or control sensors 13, 14). The amount or concentration of molecule A′ bound to control sensors 13, 14 may be used for calibration or positive controls relative to a signal generated at test sensor 11. Preferably, the amount or concentration of molecule A′ bound to control sensors 13, 14 is in a range that is relevant to concentrations of analyte A to be detected at test sensor 12.

As indicated in FIG. 4, control sensors 13, 14 may have analyte A bound thereto. However, if control sensors 13, 14 are in fluid communication with test sensor 11, there is concern regarding diffusion or movement of analyte A that may break free from control sensor 13, 14 or linker 60 or otherwise migrate to test sensor 11 where analyte A may bind with capture analyte-binding partner 40. This could result in false positive results or otherwise inaccurate results. Because molecule A′ does not bind to capture analyte-binding partner 40, the potential undesired effects of diffusion or migration and potential false positive or inaccurate results may be mitigated or prevented. Further, the amount of free A′ would likely be insignificant to the amount of conjugate.

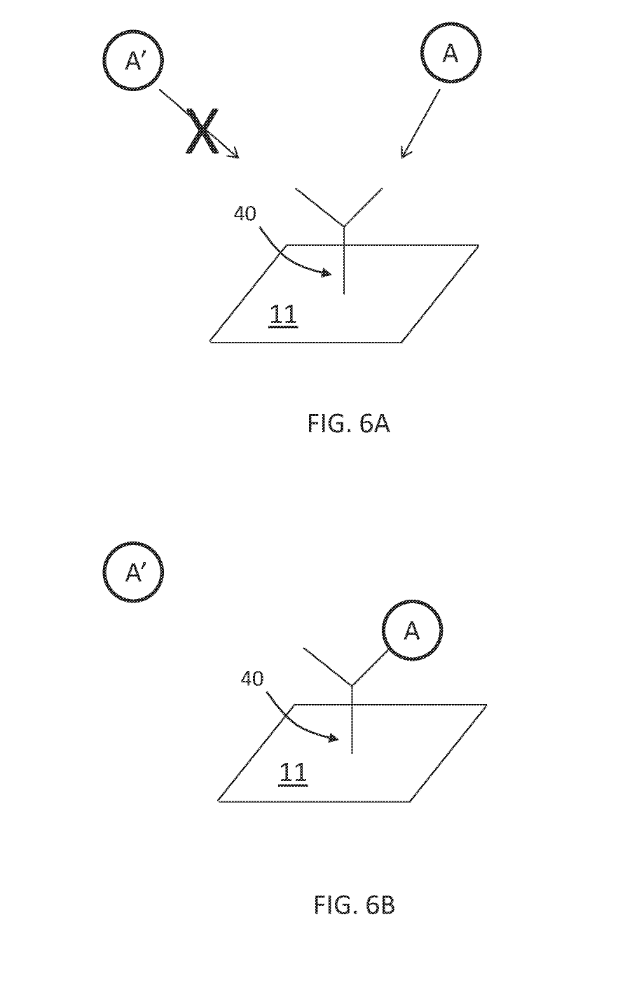

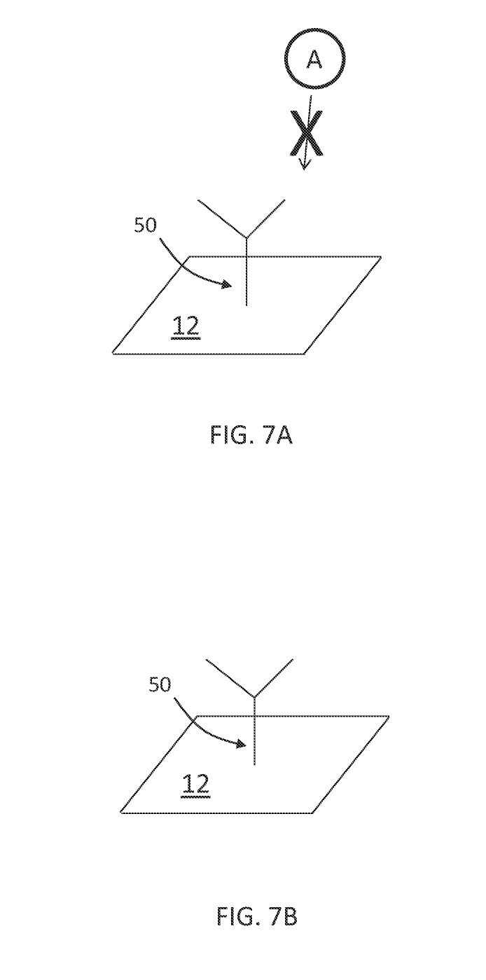

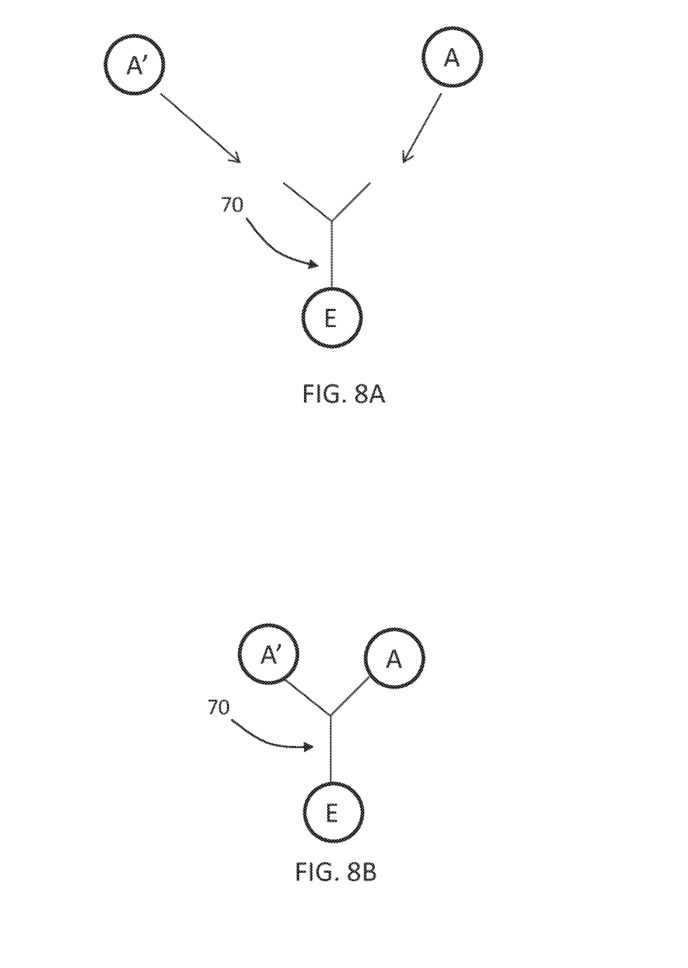

For purposes of further illustration, FIGS. 6-8 are presented. As shown in FIGS. 6A and 6B, capture analyte-binding partner 40 of test sensor 11 selectively binds analyte A, but does not bind molecule A′. As shown in FIGS. 7A and 78, molecule 50 that is similar to capture analyte-binding partner and that is bound to reference sensor 12 does not bind analyte A (and does not bind molecule A′—not shown). As shown in FIGS. 8A and 8B, conjugate analyte-binding partner 70 selectively binds to both analyte A and molecule A′ (e.g., via a common epitope or other common structural moiety). While conjugate analyte-binding partner 70 is shown as binding both analyte A and molecule A′ at the same time, it will be understood that in some case, conjugate analyte-binding partner 70 may be able to bind only one of analyte A and molecule A′ at a given time (e.g., only one binding site) but is capable of selectively binding either analyte A or molecule A′.

Any suitable capture analyte-binding component 40 may be bound to the surface of a test sensor 11. The capture analyte-binding component 40 preferably selectively binds to the analyte A of interest. By way of example, the capture analyte-binding component 40 may be selected from the group consisting of nucleic acids, nucleotide, nucleoside, nucleic acids analogues such as PNA and LNA molecules, proteins, peptides, antibodies including IgA, IgG, IgM, IgE or antibody fragments such as Fab fragments, lectins, enzymes, enzymes cofactors, enzyme substrates, enzymes inhibitors, receptors, ligands, kinases, Protein A, Poly U, Poly A, Poly lysine, triazine dye, boronic acid, thiol, heparin, polysaccharides, coomassie blue, azure A, metal-binding peptides, sugar, carbohydrate, chelating agents, prokaryotic cells and eukaryotic cells. It will be understood that the capture analyte-binding component 40 will be selected based on the analyte of interest.

Analyte A may be any suitable analyte, including for example polynucleic acids, lipids, phospholipids, polypeptides, antibodies, antigens, enzymes, enzyme substrates, a prokaryotic cell, an eukaryotic cell, a virus or virus particle, a phage or phagemid, and the like. In some embodiments, analyte A is a component of a troponin complex, such as troponin I (TnI) which is a protein present in people suffering from or who have recently suffered from a heart attack.

Any suitable analyte non-binding partner 50 that is similar to the capture analyte-binding partner 40 may be bound to the surface of reference sensor 12. Preferably, analyte non-binding partner 50 does not bind, does not selectively bind, or does not bind sufficiently high affinity to withstanding washing, analyte A. In some embodiments where analyte analog (molecule A′) is employed, analyte non-binding partner 50 preferably does not bind, or does not selectively bind, or does not bind with sufficiently high affinity to withstanding washing molecule A′. Analyte non-binding partner 50 is preferably as similar as possible to capture analyte-binding partner 40, except that it lacks the ability to bind analyte A. For example, if capture analyte-binding partner 40 is a polynucleic acid having a sequence that binds with analyte, analyte non-binding partner 50 may be a nucleic acid having a scrambled (same nucleotides in different order), non-analyte binding sequence. By way of further example, if capture analyte-binding partner 40 is an antibody, analyte non-binding partner 50 may be an antibody of the same subtype (e.g., IgG₁), except that it does not bind analyte A. Analyte non-binding partner 50 may be a type of molecule the same as or similar to capture analyte-binding partner 40. For example, analyte non-binding partner 50 may be selected from the group consisting of nucleic acids, nucleotide, nucleoside, nucleic acids analogues such as PNA and LNA molecules, proteins, peptides, antibodies including IgA, IgG, IgM, IgF, or antibody fragments such as Fab fragments, lectins, enzymes, enzymes cofactors, enzyme substrates, enzymes inhibitors, receptors, ligands, kinases, Protein A, Poly U, Poly A, Poly lysine, triazine dye, boronic acid, thiol, heparin, polysaccharides, coomassie blue, azure A, metal-binding peptides, sugar, carbohydrate, chelating agents, prokaryotic cells and eukaryotic cell.

Any suitable linker molecule 60 may be bound to a control sensor 13, 14. Linker 60 may be any suitable synthetic polymer linker, a protein, a polypeptide, a polynucleic acid, an enzyme, an antibody, or the like. Linker 60 can be any of the types of molecules listed above with regard to capture analyte-binding partner. In some embodiments, the linker is bovine serum albumin (BSA). An example of a suitable synthetic polymer is polyethylene glygol) (PEG).

Analyte A or analyte analog (molecule A′) may be bound to linker 60 in any suitable manner. For example, analyte A can be covalently bound to linker 60. It will be understood that the nature of the linker 60 and analyte A or molecule A′ will dictate, at least in part, the process employed to bind analyte A or molecule A′ to linker 60. In some embodiments (not shown), analyte A or molecule A′ are bound to the surface of a control sensor 13, 14 without the use of a linker.

Preferably, analyte analog (molecule A′) selectively binds conjugate analyte-binding partner 70 but not capture analyte-binding partner 40. Preferably, molecule A′ binds conjugate analyte-binding partner 70 in a manner similar to analyte A. For example, if analyte A is a molecule polypeptide) having multiple epitopes capable of binding multiple antibody domains e.g, capture analyte-binding partner and conjugate analyte binding partner, if the binding partners are antibodies), molecule A′ may comprise an epitope that binds conjugate analyte binding partner 70 but not capture analyte-binding partner 40.

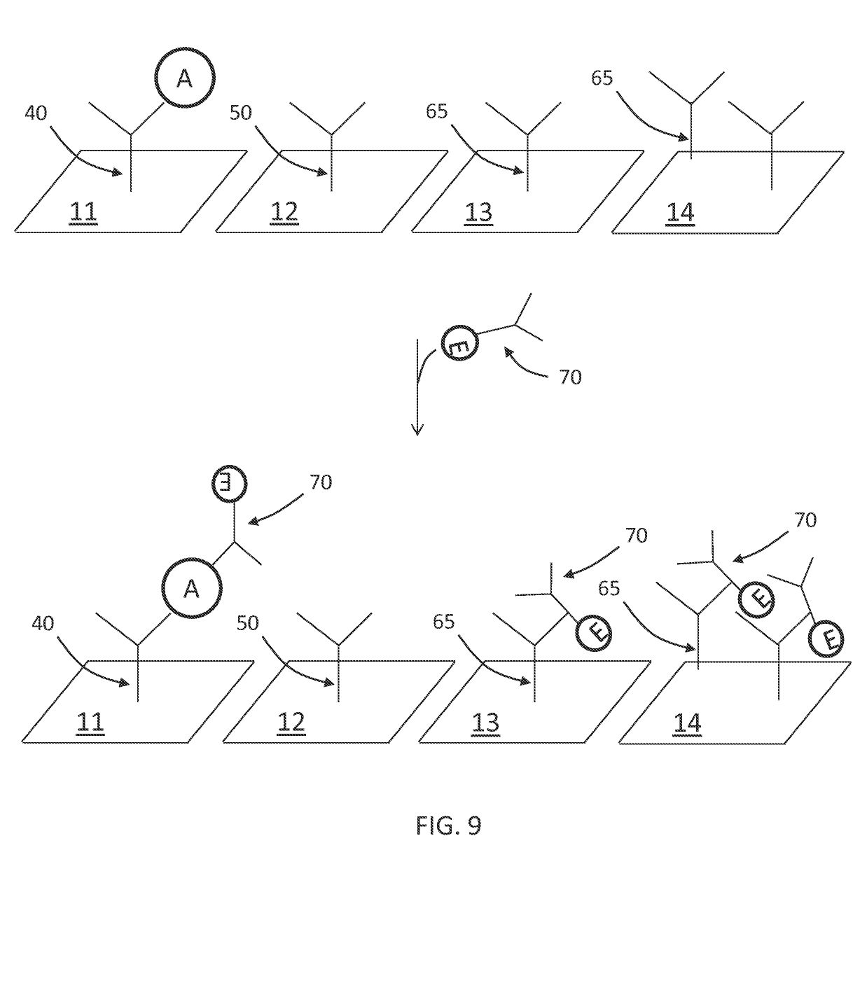

Referring now to FIG. 9 (which corresponds generally to the bottom two panels of FIG. 5), an alternative embodiment is shown where a molecule 65, which binds conjugate analyte-binding partner 70, is bound to a surface of control sensor 13, 14 (as opposed to having linker 60 bound to analyte A or molecule A′ bound to sensor). Any suitable molecule 65 that can bind conjugate analyte-binding partner 70 may be employed. It will be understood that the nature of conjugate analyte-binding partner 70 will dictate, at least in part, the nature of molecule 65 that may be used. In some embodiments, conjugate analyte-binding partner 70 comprises a mouse antibody (e.g., IgG) and molecule 65 comprises an anti-mouse antibody (e.g., anti-mouse IgG antibody).

However, it is preferred to employ a linker 60 bound to analyte A or molecule A′ that is similar to (e.g., has properties—e.g, epitope—of) analyte A (e.g., as shown in FIG. 4 or FIG. 5), as opposed to molecule 65 that binds conjugate analyte-binding partner 70 (e.g., as shown in FIG. 9). Using linker bound to analyte A or molecule A′ that is similar to analyte A because analyte A or molecule A′ will account for changes in the ability of conjugate analyte-binding partner 70 to bind analyte (and thus serve as a better control), while the use of molecule 65 may not account for such changes. In addition, in some cases (e.g., where capture analyte-binding partner is a mouse antibody and molecule is an anti-mouse antibody), unintended release of molecule 65 from surface of control sensor 13, 14 may result in erroneous response at test sensor 11 if molecule 65 binds with capture analyte-binding partner 40.

In any of the embodiments discussed or contemplated herein, any suitable conjugate analyte-binding partner 70 may be employed. Conjugate analyte-binding partner 70 includes a moiety that selectively binds analyte A (and may bind molecule A′ and may be bound by molecule 65). The moiety that selectively binds analyte A (or molecule A′) may be for example, selected from the group consisting of nucleic acids, nucleotide, nucleoside, nucleic acids analogues such as PNA and LNA molecules, proteins, peptides, antibodies including IgA, IgG, IgM, IgE or antibody fragments such as Fab fragments, lectins, enzymes, enzymes cofactors, enzyme substrates, enzymes inhibitors, receptors, ligands, kinases, Protein A, Poly U, Poly A, Poly lysine, triazine dye, boronic acid, thiol, heparin, polysaccharides, coomassie blue, azure A, metal-binding peptides, sugar, carbohydrate, chelating agents, prokaryotic cells and eukaryotic cell.

Conjugate analyte-binding partner 70 preferably also includes a moiety E that is a signaling moiety or signal facilitating moiety. Examples of suitable signaling moieties or signal facilitating moieties include moieties that emit or display color, fluorescence, isotope, light, and the like. In some embodiments, the moiety E is an activatable polymerization initiator, such as a photoinitiator, a chemical initiator, or a thermoinitiator. The polymerization initiator may be activated in the presence of one or more monomers to cause a polymer to graft from the second molecular recognition component. As the growing polymer is attached to the surface of the sensor, mass is added to the surface of the sensor. In some embodiments, moiety E comprises an enzyme. In some embodiments, the enzyme is capable of converting a substrate that is soluble in the assay environment to an insoluble product that precipitates on the surface of the sensor. Examples of suitable enzymes include alkaline phosphatase (ALP), horse radish peroxidase (HRP), beta galactosidase, and glucose oxidase. Moieties E that are configured to add mass to the sensor are preferably employed with mass sensing sensors, such as TFBARs.

Examples of enzyme/substrate systems that are capable of producing an insoluble product which is capable of accumulating on a surface include alkaline phosphatase and 5-bromo-4-chloro-3-indolylphosphate/nitro-blue tetrazolium chloride (BCIP/NBT). The enzymatically catalyzed hydrolysis of BCIP produces an insoluble dimer, which may precipitate on the surface of the sensors. Other analogous substrates having the phosphate moiety replaced with such hydrolytically cleavable functionalities as galactose, glucose, fatty acids, fatty acid esters and amino acids can be used with their complementary enzymes. Other enzyme/substrate systems include peroxidase enzymes, for example horse radish peroxidase (HRP) or myeloperoxidase, and one of the following: benzidene, benzidene dihydrochloride, diaminobenzidene, o-tolidene, o-dianisidine and tetramethyl-benzidene, carbazoles, particularly 3-amino-9-ethylcarbazole, and various phenolic compounds all of which have been reported to form precipitates upon reaction with peroxidases. Also, oxidases such as alphahydroxy acid oxidase, aldehyde oxidase, glucose oxidase, L-amino acid oxidase and xanthine oxidase can be used with oxidizable substrate systems such as a phenazine methosulfate-nitriblue tetrazolium mixture.

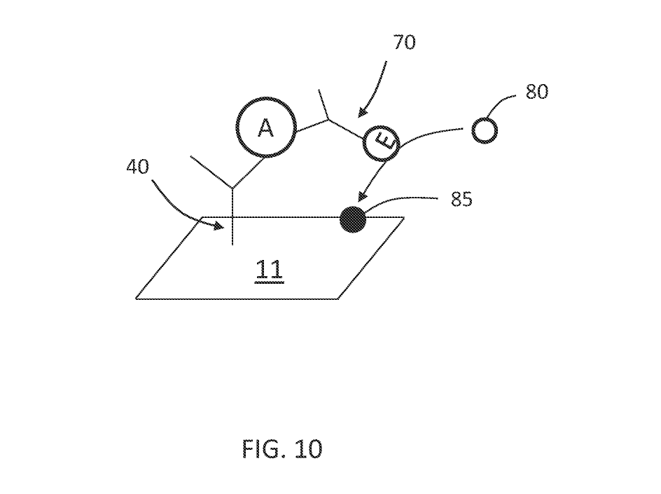

Referring now to FIG. 10, a schematic drawing of a test sensor 11 is depicted. The test sensor 11 has a surface to which a capture analyte-binding partner is bound. Analyte A is bound to the capture analyte-binding partner. Conjugate analyte-binding partner 70 having element E, such as an enzyme, is bound to analyte A. Element E, such as an enzyme, converts amplification precursor 80, such as a substrate, to insoluble molecule 85 that precipitates on the surface of the sensor 11 thereby adding mass to the surface of the sensor, which can be advantageously employed to enhance sensitivity of assays employing mass sensors such as TFBARs.

In some embodiments, a piezoelectric-based specific binding assay includes signal amplification of the reaction between a molecular recognition component and its target analyte to provide a more sensitive assay. One such example is presented in U.S. Pat. No. 4,999,284 issued to Ward on 12 Mar. 1991, which patent is hereby incorporated herein by reference in its entirety to the extent that it does not conflict with the disclosure presented herein. The '284 patent discloses a method using a quartz crystal microbalance assay, in which the binding of analyte to a surface on or near a quartz crystal microbalance (QCM) is detected by a conjugate that includes an enzyme. The enzyme is capable of catalyzing the conversion of a substrate to a product capable of accumulating on or reacting with a surface of the QCM leading to a mass change and, hence, a change in resonant frequency. Another example is presented in PCT Patent Application No. PCT/US2014/027743, filed on Mar. 14, 2014, entitled THIN FILM BULK ACOUSTIC RESONATOR WITH SIGNAL ENHANCEMENT, naming Rapid Diagnostek, Inc. as applicant, and having attorney docket number 468.00020201, which PCT patent application is hereby incorporated herein by reference in its entirety to the extent that it does not conflict with the disclosure presented herein. This PCT patent application discloses, among other things, signal amplification for thin film bulk acoustic resonator (TFBAR) sensors.

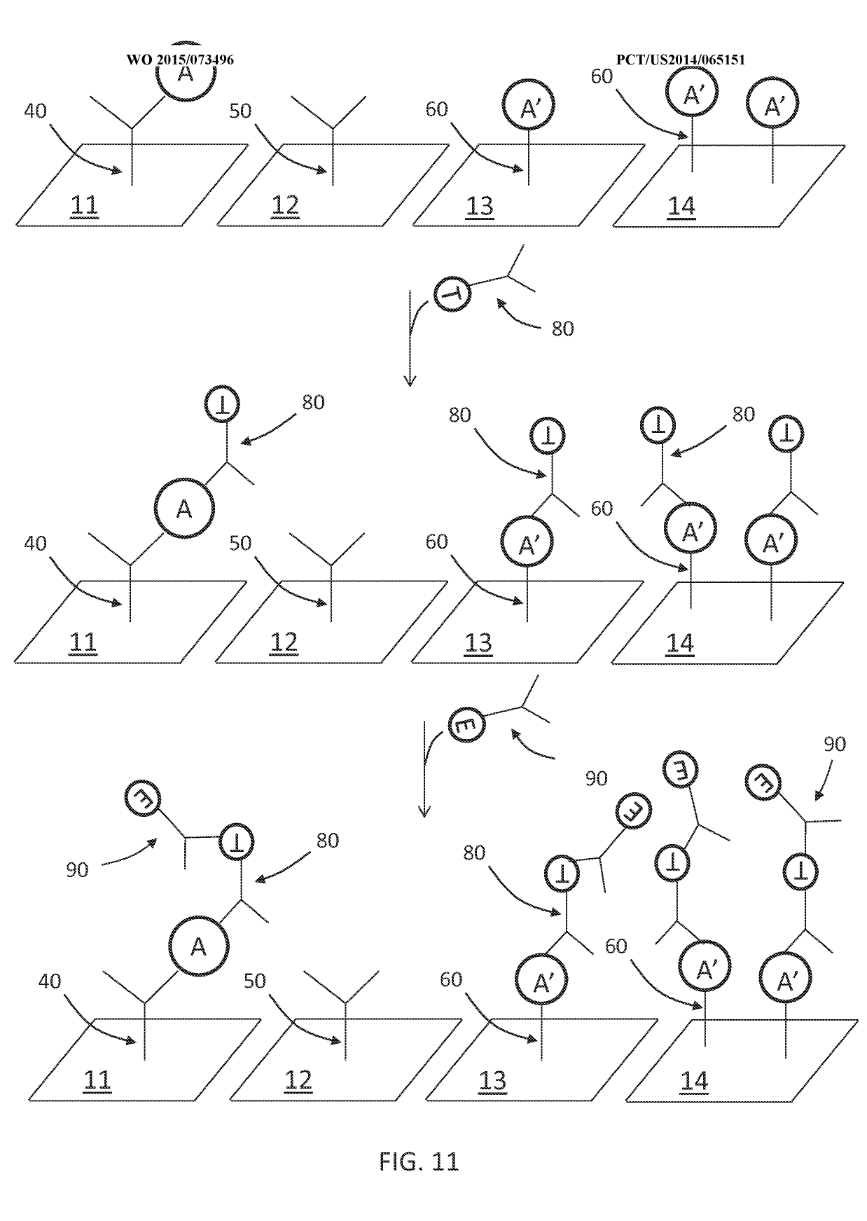

While the scheme depicted in, for example, FIGS. 9-10 show conjugate analyte-binding partner 70 as having bound moiety E, it will be understood that additional molecules may be employed to bind moiety E to conjugate analyte-binding partner 70. In some embodiments, conjugate analyte-binding partner includes a tag that is selectively recognized by a tag-binding partner that includes the moiety E. For example and with reference to FIG. 11, an embodiment of an assay scheme employing such components is shown.

The top panel in FIG. 11 is the same as or similar to the top panel depicted in FIG. 5, and includes a test sensor 11 to which a capture analyte-binding partner 40 is bound, and to which an analyte A is bound (after addition of test sample). Reference sensor 12 has an analyte non-binding partner bound to its surface and thus has no bound analyte A. Control sensors 13 and 14 have differing known amounts of molecule A′ bound to their respective surfaces via linkers 60. A tag T-linked conjugate analyte-binding partner 80 is introduced to sensors 11, 12, 13, 14 (e.g., via flow path). Tag T-linked conjugate analyte-binding partner 80 selectively binds to analyte A and molecule A′ and thus binds to test sensor 11 and control sensors 13 and 14 but not reference sensor 12 (see middle panel). A moiety E-linked tag-binding partner 90 is then introduced to sensors 11, 12, 13, 14 (e.g., via flow path). Moiety E-linked tag-binding partner 90 selectively binds to tag T. Accordingly, Moiety E-linked tag-binding partner 90 is bound to test sensor 11 and control sensors 13 and 14 but not reference sensor 12 (see bottom panel). As discussed above, moiety E is a signaling moiety or signal facilitating moiety.

Tag T can be any suitable tag that is selectively bound by a tag-binding partner. Examples of suitable tags include a streptavidin tag; a biotin tag; a chitin binding protein tag; a maltose binding protein tag; a glutathione-S-transferase tag; a poly(His) tag; an epitope tag such as a Myc tag, a HA tag, or a V5 tag; or the like. By way of example, tag-linked conjugate analyte-binding partner 80 can be biotinylated, and moiety E-linked tag-binding partner 90 may be conjugated to streptavidin; or vice-versa. By way of further example, tag-linked conjugate analyte-binding partner 80 and moiety E-linked tag-binding partner 90 can be a polyhistidine(His) tag, and the other can be, for example, a nickel or copper chelator, such as as iminodiacetic acid (Ni-IDA) and nitrilotriacetic acid (Ni-NTA) for nickel and carboxylmethylaspartate (Co-CMA) for cobalt, which the poly(His) tag can bind with micromolar affinity. Generally nickel-based resins have higher binding capacity, while cobalt-based resins offer the highest purity. By way of yet another example, one of tag-linked conjugate analyte-binding partner 80 and moiety E-linked tag-binding partner 90 can be a glutathione-S-transferase (GST) tag, and the other can be glutathione. For yet another example, one of tag-linked conjugate analyte-binding partner 80 and moiety E-linked tag-binding partner 90 can be a maltose binding protein tag, and the other can be amylose or maltose. As another example, one of tag-linked conjugate analyte-binding partner 80 and moiety E-linked tag-binding partner 90 can be a chitin binding protein tag, and the other can be chitin. It will be understood that above-presented binding partners are merely examples of high affinity binding partners that may be conjugated to an analyte-binding partner or a moiety E and that other binding partners are contemplated herein. Further, it will be understood that more than one set of binding partners may be employed to link moiety E to analyte A or analyte analog (molecule A′).

Binding partners may be conjugated to analyte-binding partners or moieties E through any suitable technique. For example, chemical conjugation or recombinant techniques may be employed to link a binding partner to analyte-binding partners or moieties E, as appropriate. Such techniques are well known to those of skill in the art. For example, a heterobifunctional cross linker utilizing NHS-ester and maleimide functional groups may be employed as known to those of skill in the art.

In some embodiments, a competition assay is employed with the methods, devices or systems described herein. In a competition assay, the analyte in a sample competes for binding with the capture analyte-binding partner immobilized on the surface of the test sensor. In some embodiments, the analyte competes for binding with a tag-linked analyte that competes with the capture analyte-binding partner for the same binding site as the analyte. The tag-linked analyte may include a variant or derivative of the analyte. The variant or derivative can be a variant or derivative that is selectively recognizable by the capture analyte-binding partner. In some situations, it may be desirable that the variant or derivative analyte have an affinity for the capture analyte-binding partner or conjugate analyte-binding partner that is different than the affinity of the non-tag-linked analyte. The variant or derivative of the analyte may be a variant or derivative that allows for ease of manufacture of the tag-linked analyte. For example, the tag-linked analyte may comprise a recombinant polypeptide, etc. A moiety E-linked tag-binding partner, which can be as discussed above, can be used to generate a signal measurable by the test sensor in some embodiments. Signal reduction is proportional with an increase in analyte concentration as the analyte competes with or displaces tag-linked analyte binding to the test sensor, which thereby reduces binding of moiety E-linked tag-binding partner and resulting signal.

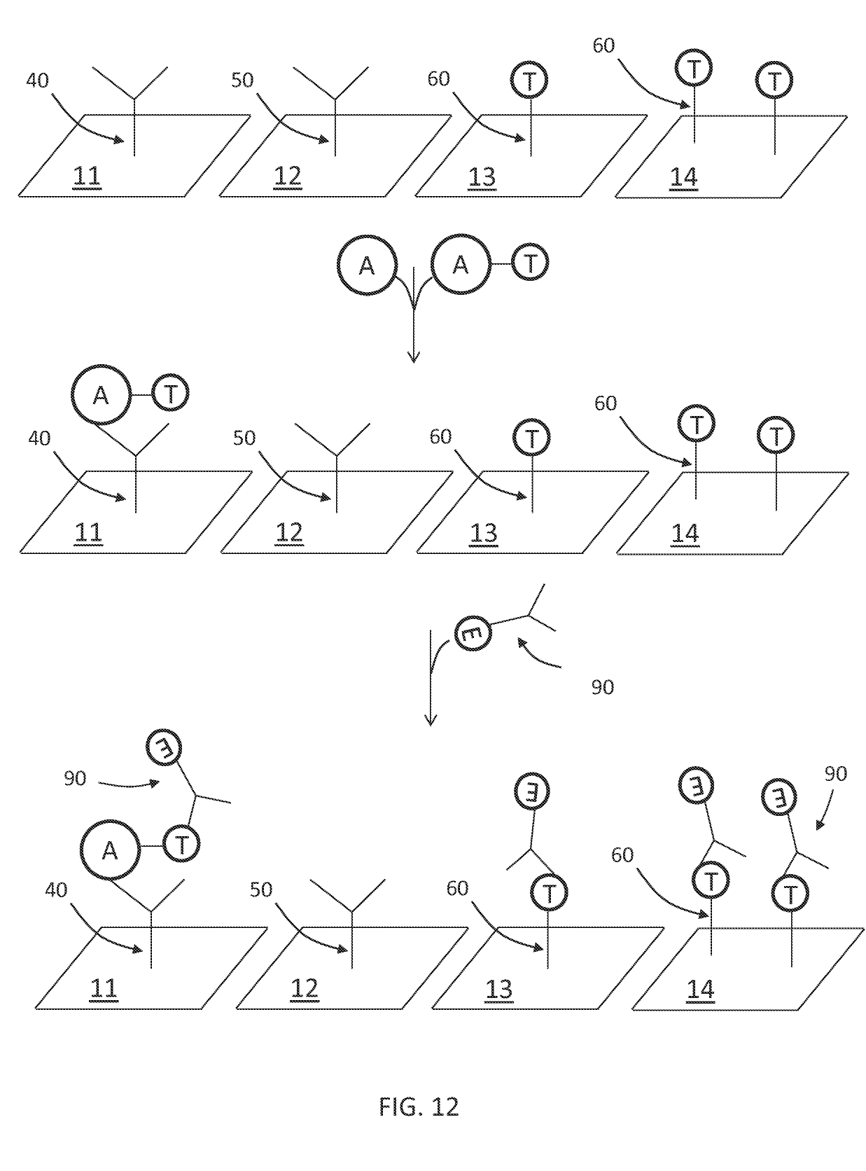

An embodiment of an assay scheme for a competition assay is depicted in FIG. 12. In the depicted embodiment, a capture analyte-binding partner 40, which can be as described above, is immobilized on a surface of test sensor 11. An analyte non-binding partner 50, which may be as described above, is immobilized on a surface of reference sensor 12. Tag T, which can be as discussed above, is immobilized on a surface of control sensors 13, 14 via linker 60, which can be as discussed above. In some embodiments (not shown), tag T can be immobilized directly on the surface of the control sensors 13, 14 without linker 60. In some embodiments (not shown), an analyte A-liked tag T (A-T), rather than tag T, is immobilized on a surface of control sensor 13, 14 either directly or via linker 60. A sample, which may contain analyte A, and analyte A-linked tag T (A-T) are introduced to surfaces of sensors 11, 12, 13, 14; e.g., via a flow path. Analyte A and analyte A-linked tag T compete for binding at capture-analyte binding partner 40, while neither analyte A nor analyte A-linked tag T (A-T) binds analyte non-binding partner 50, Moiety E-linked tag binding partner 90, which may be as described above, is introduced to surfaces of sensors of sensors 11, 12, 13, 14 and binds to tag T of analyte linked tag (A-T) that is bound to capture analyte binding partner 40 immobilized on the surface of the test sensor 11 and binds to tag T that is bound to linker 60 immobilized on surfaces of control sensors 13, 14. Signals generated by moiety E at test sensor 11, reference sensor 12, and control sensors 13, 14 can be compared to determine the amount of analyte present in the sample.

All scientific and technical terms used herein have meanings commonly used in the art unless otherwise specified. The definitions provided herein are to facilitate understanding of certain terms used frequently herein.

As used herein, the singular forms “a”, “an”, and “the” encompass embodiments having plural referents, unless the content clearly dictates otherwise.

As used herein, “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising,” and the like.

The words “preferred” and “preferably” refer to embodiments of the methods, devices, systems, etc. described herein that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

As used herein, “sensor” means a device or components thereof that detects or measures a physical property. The device may include activation circuitry and components, measurement circuitry and components and analyte binding sites.

### EXAMPLE

The following non-limiting prophetic example serves to describe more fully the manner of using the above described sensors, methods, devices and systems. It is understood that this prophetic example in no way serve to limit the scope of this disclosure or claims that follow, but rather is presented for illustrative purposes.

A sensor device may include sensors, or analyte-binding or analyte-nonbinding components thereof, in a row, spaced roughly 50 microns apart, on a single chip (printed circuit board). Four sensors, a test sensor, a reference sensor, and two control sensors, may be employed to provide a single high quality result. The device may include assay specific spotted and dried capture reagents on the sensors and assay specific liquid enzyme-antibody conjugate (complimentary to the capture antibody) in a carousel. A sample (e.g., a patient sample) can be added to the cartridge, mixed with liquid reagents in the carousel then passed over the sensor. Depending on the assay a variety of steps are possible but ultimately all assays generate a response from the sensor(s). Finally, a sensor response is transformed into a quantitative result by interpolation off a calibration curve, stored on the instrument, which can be generated at the factory. To generate a reliable result this quantification approach may require periodic recalibration to account for conjugate enzymatic degradation (it is believed that the dried capture reagents will be stable over the shelf-life of the product). This recalibration can be accomplished by running an external calibration solution and adjusting the calibration to generate the correct (known) result; however this approach is time/reagent consuming and does not guarantee the instrument/assay is always in current calibration. The concept being proposed here obviates the need to run external calibrators, improves the fidelity of the result by multiple calibrations at the time of testing, and ensures the instrument/assay calibration is current.

The proposed concept is to utilize one sensor as the test sensor. In the case of TnI the test sensor will be coated with capture reagents (antibodies) specific to TnI. Another sensor, the reference sensor will be coated with an irrelevant molecule that is as similar to the specific capture reagents as possible but does not interact with the antigen of interest (in this case TnI). Typically the reference sensor is coated with an irrelevant antibody of the same isotype (if possible) of the capture antibody. A third sensor (control sensor) will be coated with a low level (at or near clinical decision point) of antigen (TnI in this case) and the fourth sensor (control sensor) will be coated with slightly more (most likely) or possibly less antigen. Collectively the reference sensor and the two antigen (control) sensors are termed the Control Sensors. During an assay conjugate will bind to the test sensor (if antigen is present in the sample), and the two sensors coated with varying amounts of antigen. No conjugate should bind to the reference sensor. Utilizing the response from Control Sensors a three point calibration (i.e. negative, low, and medium) adjustment can be done, tier every test, at the time of testing. Furthermore, the Control Sensors can be used as internal quality control for each assay as the signal generated on each should fall with defined limits. Failure of Control Sensors responses to fall within predetermined limits could be used to warn clinicians of potentially erroneous results (flag) or even prevent a result from being reported.

One of the potential pitfalls of this approach would be antigen leaching from Control Sensors. For example, in a TnI assay if TnI was spotted onto the positive Control Sensors then any TnI that may leach off of these Control Sensors could conceivably be captured by the test sensor and generate an elevated response. The described leaching scenario could generate a false-positive. To eliminate this possibility we are utilizing positive Control Sensors that are coated with molecule (peptide) that only contains the epitope of the conjugate antibody and is not capable of (or configured to) binding to the test sensor. Using peptide epitopes to the conjugate antibody is a superior approach to utilizing something like an anti-mouse IgG antibody (a typical approach in lateral flow assays assuming the conjugate antibody is a mouse antibody) because the peptide epitopes will account for changes in conjugate enzyme and antibody functionality while an anti-mouse control sensor would only account for changes in conjugate enzyme functionality. Also, much like antigen, leaching of an anti-mouse IgG antibody from a positive control sensors could interact with capture antibody on the test sensor (if it is a mouse IgG) and generate an erroneous response.

Utilization of this concept should generate a higher quality result then those obtained in existing devices, such as the high-throughput analyzers used in a central lab, which do not (and cannot) run multiple controls on each sample.

Thus, methods, systems, and devices for SENSORS HAVING INTERNAL CALIBRATION OR POSITIVE CONTROLS are described. Various modifications and variations of the methods, systems and devices described herein will be apparent to those skilled in the art without departing from the scope and spirit of the disclosure. Although this disclosure has been described in connection with specific preferred embodiments, it should be understood that the methods, devices and systems as claimed should not be unduly limited to such specific embodiments. Indeed, various modifications of the described modes for carrying out aspects of the invention which are apparent to those skilled in related fields are intended to be within the scope of the following claims.

The teachings presented herein may be employed with any device or system having multiple sensors or detection zones that are spatially separated and that are in fluid communication and that otherwise are within the scope or spirit of the present disclosure.

1. A device for detecting an analyte in a sample, comprising:
one or more test sensors configured to bind the analyte;
one or more control sensors comprising bound analyte or analyte analog, wherein each control sensor comprises a known amount of bound analyte or analyte analog prior to performing an assay to detect the analyte in the sample;
a fluid flow path for carrying the sample, wherein the one or more test sensors and the one or more control sensors are positioned along and in communication with the fluid flow path; and
control electronics operably coupled to the one or more test sensors and the one or more control sensors, wherein the control electronics are configured to compare signals generated from the one or more test sensors and the one or more control sensors to determine an amount of analyte present in the sample.

2. A device according to claim 1, wherein:
each of the one or more test sensors comprises a thin film bulk acoustic resonator (TFBAR) comprising a surface to which an analyte-binding partner is immobilized; and
each of the one or more control sensors comprises a TFBAR comprising a surface to which the analyte or the analyte analog is immobilized.

3. A device according to claim 2, wherein each of the TFBARs of the one or more test sensors and the one or more control sensors has a resonance frequency of 900 MHz or greater.

4. A device according to claim 2, wherein the control electronics comprise:
actuation circuitry configured to drive each of the TFBARs of the one or more test sensors and the one or more control sensors into oscillating motion;
measurement circuitry arranged to be coupled to each of the TFBARs of the one or more test sensors and the one or more control sensors and configured to measure one or more output signals of each of the TFBARs representing resonance characteristics of the oscillating motion of each of the TFBARs; and
a controller operatively coupled with the actuation and measurement circuitry.

5. A device according to claim 1, wherein the device comprises a plurality of control sensors, each having varying known amounts of bound analyte or analyte analog.

6. A device according to claim 1, wherein the analyte is troponin I (TnI).

7. A device according to claim 1, further comprising one or more reference sensors, each being configured not to bind the analyte and not comprising bound analyte or bound analyte analog, wherein the one or more reference sensors are positioned along and in communication with the flow path, and wherein the control electronics are configured to compare signals generated from the one or more test sensors, the one or more reference sensors, and the one or more control sensors to determine an amount of analyte present in the sample.

8. A device according to claim 7, wherein the one or more reference sensors each comprise a thin film bulk acoustic resonator (TFBAR) comprising a surface to which an analyte-nonbinding partner is immobilized.

9. A device according to claim 8, wherein each of the TFBARs of the one or more reference sensors have a resonance frequency of 900 MHz or greater.

10. A device according to claim 7, wherein the control electronics comprise:
actuation circuitry configured to drive each of the TFBARs of the one or more test sensors, the one or more reference sensors, and the one or more control sensors into oscillating motion;
measurement circuitry arranged to be coupled to each of the TFBARs of the one or more test sensors, the one or more reference sensors, and the one or more control sensors and configured to measure one or more output signals of each of the TFBARs representing resonance characteristics of the oscillating motion of each of the TFBARs; and
a controller operatively coupled with the actuation and measurement circuitry.

11. A sensor assembly for use with a device for detecting an analyte in a sample, wherein the device has a flow path through which the sample is configured to flow, the sensor assembly comprising:
one or more test sensor comprising a thin film bulk acoustic resonator (TFBAR), each having a surface to which an analyte-binding partner is immobilized prior to performing an assay to detect the analyte in the sample; and
one or more control sensors comprising a TFBAR, each having a surface to which the analyte or the analyte analog is immobilized,
wherein the sensor assembly is configured to be operably coupled with the device for detecting the analyte in the sample, and wherein the sensor assembly is configured to be at least partially inserted into the device such that the one or more test sensor and the one or more control sensor are positioned in and in communication with the flow path of the device.

12. A sensor assembly according to claim 11, further comprising a printed circuit board, wherein the one or more test sensors and the one or more control sensors are disposed on the printed circuit board.

13. A sensor assembly according to claim 11, wherein the one or more test sensors and the one or more control sensors are disposed in a cartridge configured to be at least partially inserted into the device.

14. A sensor assembly according to claim 11 any, wherein each of the TFBARs of the one or more test sensors and the one or more control sensors has a resonance frequency of 900 MHz or greater.

15. A sensor assembly according to claim 11, wherein the sensor assembly comprises a plurality of control TFBAR sensors, each having varying known amounts of bound analyte or analyte analog immobilized thereto.

16. A sensor assembly according to claim 11, wherein the analyte is troponin I (TnI).

17. A sensor assembly according to claim 11, further comprising one or more reference sensors, each comprising a TFBAR having a surface to which an analyte-nonbinding partner is immobilized, wherein the sensor assembly is configured to be at least partially inserted into the device such that the one or more test sensor, the one or more reference sensors, and the one or more control sensor are positioned in and in communication with the flow path of the device.

18. A sensor assembly according to claim 17, wherein each of the TFBARs of the one or more reference sensors has a resonance frequency of 900 MHz or greater.

19. A method for determining an amount of an analyte in a sample, comprising:
introducing (i) the sample or (ii) the sample and a tag-linked analyte molecule to a fluid flow path disposed across at least a portion of one or more test sensors and one or more control sensors,
wherein each of the one of more test sensors, in the flow path, comprises a surface to which an analyte-binding partner is immobilized, and
wherein each of the one or more control sensors, in the flow path, comprises a surface to which the analyte, the tag-linked analyte molecule, an analyte analog, a tag-linked analyte analog, or a tag is immobilized prior to introducing the sample;
measuring an amount, or indicator thereof, of the analyte, the tag-linked analyte, or the tag bound to the one or more test sensors and the one or more control sensors; and
comparing the amount of analyte, tag-linked analyte, tag or indicator thereof bound to the one or more test sensors and the one or more control sensors to determine the amount of analyte in the sample.

20. A method according to claim 19, further comprising:
introducing a signal enhancement element-linked analyte recognition component or a tag-linked analyte recognition component to the flow path disposed across the one or more test sensors and the one or more control sensors;
introducing a signal enhancement element-linked tag recognition component to the flow path if the tag-linked analyte recognition component is introduced to the flow path; and
introducing an amplification precursor to the flow path under conditions such that the amplification element converts the amplification precursor to a molecule that adds mass at (a) the surface of the one or more test sensors if the amplification element is bound to the surface of the test sensor, or (b) the one or more control sensors if the amplification element is bound to the surface of the control sensor.

21. A method according to claim 19, wherein the TFBARs of the test sensor, the reference sensor and the control sensor have a resonance frequency of 900 MHz or greater.

22. A method according to claim 19, wherein the analyte is troponin I (TnI).

23. A method according to claim 19,
wherein one or more reference sensors, each comprising a surface to which an analyte-nonbinding partner is immobilized, are positioned along and in communication with the flow path,
wherein introducing (i) the sample or (ii) the sample and the tag-linked analyte molecule to the fluid flow path causes the sample or the sample and the tag-linked analyte molecule to flow across the surface of each of the one or more reference sensors,
further comprising measuring an amount, or indicator thereof, of the analyte, the tag-linked analyte, or the tag bound to the one or more reference sensors; and
comparing the amount of analyte, tag-linked analyte, tag or indicator thereof bound to the one or more test sensors, the one or more reference sensors, and the one or more control sensors to determine the amount of analyte in the sample.
